(19) United States
(12) Patent Application Publication
(10) Pub. No.: US 2008/0154310 A1
(43) Pub. Date: Jun. 26, 2008

(54) Reinforced orthopedic plate

(75) Inventors: Stephen Edward White, Germantown, TN (US); Wilder Companioni, Cordova, TN (US)

Correspondence Address: KRIEG DEVAULT LLP, ONE INDIANA SQUARE, SUITE 2800, INDIANAPOLIS, IN, 46204-2709, US

(73) Assignee: Warsaw Orthopedic, Inc.

(21) Appl. No.: 11/643,158

(22) Filed: Dec. 21, 2006

(51) Int. Cl. A61B 17/58; A61B 17/56

(52) U.S. Cl. 606/280; 606/298

(57) ABSTRACT

A reinforced orthopedic plate formed of a non-metallic material and defining a primary thickness profile and including an elongate reinforcement portion defining a localized increased thickness profile to strengthen the orthopedic plate.

### FIELD OF THE INVENTION

The present invention relates generally to treatment of the spinal column, and more particularly relates to a reinforced orthopedic plate for stabilizing a portion of the spinal column.

### BACKGROUND

It is well know that orthopedic plates may be engaged to adjacent portions of bone via anchors or fasteners to provide stabilization and support of the bone portions. Such plates are commonly formed of metallic materials including titanium, stainless steel and metallic alloys.

Alternatively, non-metallic materials are sometimes used, including polymer-based materials and composite materials. However, non-metallic materials generally have reduced mechanical properties (e.g., strength, rigidity, etc.) compared to metallic materials. As a result, plates formed of non-metallic materials are typically formed via the use a greater amount of plate material (e.g., increased material thickness) to compensate for the reduction in mechanical properties. However, the use of a greater amount of plate material may not be desirable in instances where the spinal plate is formed of a resorbable material as this would significantly add to the volume of material to be resorbed into the body. Additionally, the use of a greater volume of plate material may also increase the overall size, weight and profile of the spinal plate, the likes of which may be problematic in instances where such properties and characteristics should be minimized.

Thus, there remains a need for an improved orthopedic plate for stabilizing a portion of the spinal column. The present invention satisfies this need and provides other benefits and advantages in a novel and unobvious manner.

### SUMMARY

The present invention relates generally to treatment of the spinal column, and more particularly relates to a reinforced orthopedic plate for stabilizing a portion of the spinal column. While the actual nature of the invention covered herein can only be determined with reference to the claims appended hereto, certain forms of the invention that are characteristic of the preferred embodiments disclosed herein are described briefly as follows.

In one form of the present invention, an orthopedic stabilization device is provided, including a plate formed of a non-metallic material and defining a primary thickness profile, with the plate having an elongate reinforcement portion defining a localized increased thickness profile to strengthen the plate.

In another form of the present invention, an orthopedic stabilization device is provided, including a plate formed of a non-metallic material and defining a primary material thickness, with the plate having an elongate reinforcement portion defining a localized increased material thickness to strengthen the plate.

In another form of the present invention, an orthopedic stabilization device is provided, including a plate formed of a non-metallic material and defining a primary material thickness between oppositely-facing first and second surfaces, with the plate including at least one elongate surface projection formed integral with the plate to define a unitary, single-piece plate structure and extending along a dimension of one of the first and second surfaces to strengthen the plate.

It is one object of the present invention to provide a reinforced orthopedic plate for stabilizing a portion of the spinal column. Further objects, features, advantages, benefits, and aspects of the present invention will become apparent from the drawings and description contained herein.

### BRIEF DESCRIPTION OF THE DRAWINGS

FIG. 1 is an anterior view of the cervical region of the spinal column showing a reinforced orthopedic plate according to one embodiment of the present invention secured to two cervical vertebrae.

FIG. 2 is a side perspective view of the reinforced orthopedic plate shown in FIG. 1.

FIG. 3 is a top plan view of the reinforced orthopedic plate shown in FIG. 1.

FIG. 4 is a cross-sectional view of the reinforced orthopedic plate shown in FIG. 1, as taken along line 4-4 of FIG. 3.

FIG. 5 is a side perspective view of a reinforced orthopedic plate according to another embodiment of the present invention.

FIG. 6 is a side perspective view of a reinforced orthopedic plate according to another embodiment of the present invention.

### DESCRIPTION OF THE PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended, and that alterations and further modifications to the illustrated devices and/or further applications of the principles of the invention as illustrated herein are contemplated as would normally occur to one skilled in the art to which the invention relates.

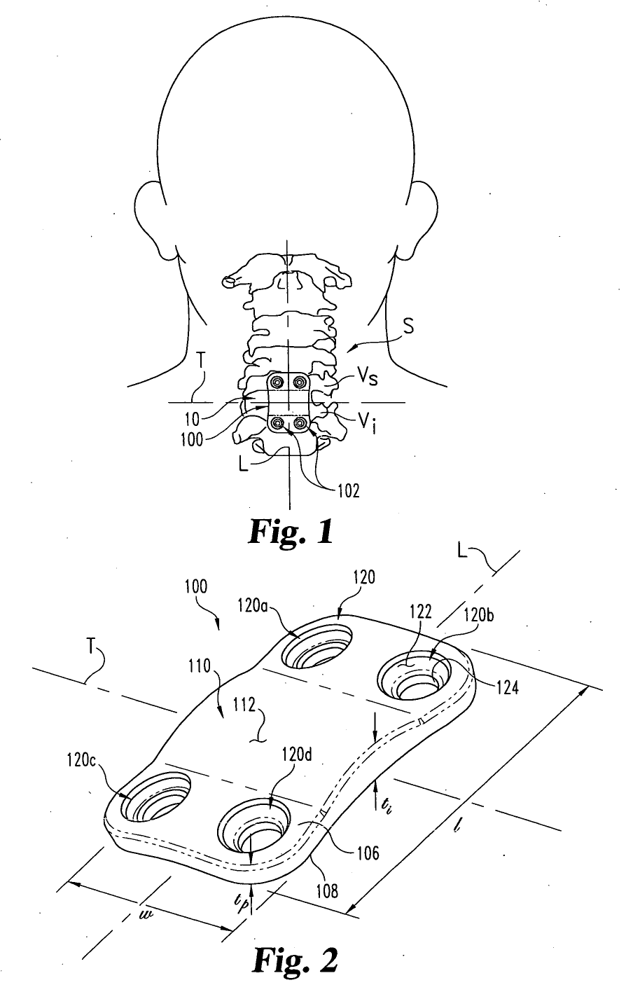

Referring to FIG. 1, shown therein is a spinal stabilization system 10 according to one embodiment of the present invention for stabilizing at least a portion of the spinal column S. The stabilization system 10 is shown attached to the cervical region of the spinal column, extending across two adjacent superior and inferior cervical vertebrae Vₛ, Vᵢ. A graft or implant (not shown) may be positioned within the disc space between the adjacent vertebrae Vₛ, Vᵢ to promote fusion. Although the stabilization system 10 is illustrated for use in association with the cervical region of the spine, it should be understood that the stabilization system 10 may also be utilized in other areas of the spine, including the thoracic, lumbar, lumbo sacral and sacral regions of the spine. It should also be understood that the stabilization system 10 can extend across any number of vertebrae, including three or more vertebrae. Additionally, although the stabilization system 10 is shown as having application in an anterior approach, the stabilization system 10 may alternatively be applied in other surgical approaches, such as, for example, a posterior approach. Furthermore, although the stabilization system 10 is shown as having application in the spinal field, it should be understood that the stabilization system 10 may alternatively be used in other orthopedic fields, including applications involving the hip, knee, shoulder, elbow, long bones, and any other orthopedic application that would occur to one of skill in the art.

The stabilization system 10 generally includes an elongate member 100 sized to span a distance between two or more vertebrae, and a plurality of bone anchors 102 for securing the elongate member 100 to the vertebrae. As will be discussed in detail below, in the illustrated embodiment, the elongate member 100 is configured as a reinforced orthopedic plate, and more particularly a reinforced spinal plate. However, although the reinforced elongate member 100 has been illustrated and described as a plate, it should be understood that the elongate member may alternatively be configured as a rod or any other type of elongate element for use in association with an orthopedic stabilization system. It should also be understood that any number of reinforced spinal plates 100, including two or more plates 100, may be used to stabilize the spinal column.

In the illustrated embodiment of the invention, the bone anchors 102 are configured as bone screws. However, other types of bone anchors are also contemplated for use in association with the spinal plate 100 including, for example, bolts, pins, staples, hooks or any other suitable anchoring device know to those of skill in the art. Bone screws suitable for use with the present invention are disclosed in U.S. Pat. No. 6,293,949 to Justis et al. and U.S. Pat. No. 6,152,927 to Farris et al., the contents of which are hereby incorporated by reference in their entirety. In the illustrated embodiment of the invention, a pair of bone screws 102 are used to anchor the reinforced spinal plate 100 to each of the vertebrae Vₛ, Vᵢ. However, in other embodiments a single bone screw or three or more bone screws may be used to anchor the reinforced spinal plate 100 to each of the vertebrae Vₛ, Vᵢ.

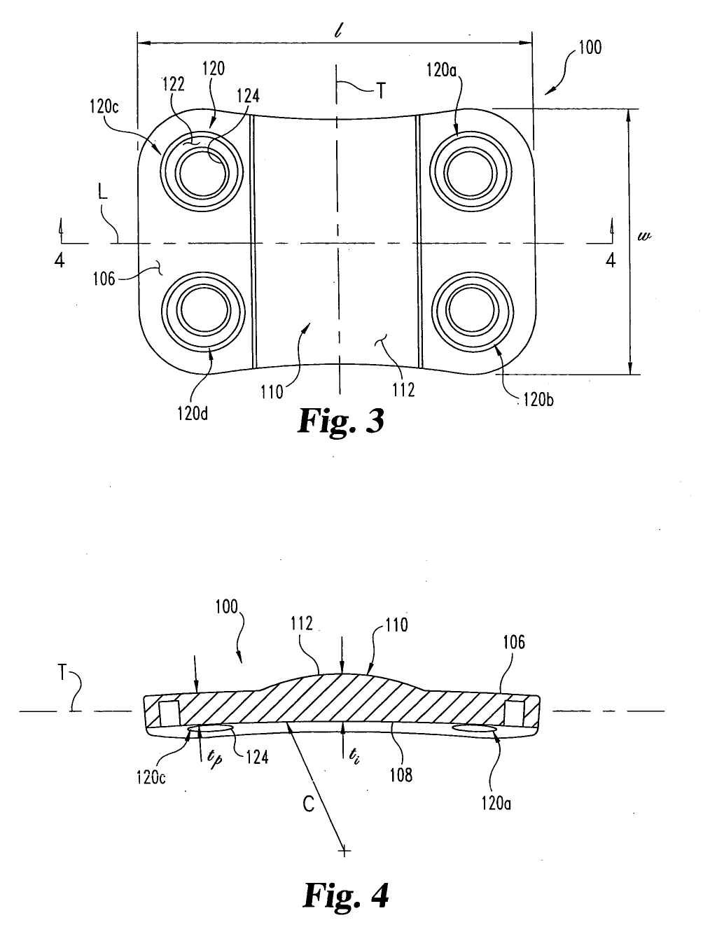

Referring to FIGS. 2-4, shown therein are further details regarding the reinforced spinal plate 100. The spinal plate 100 has a plate length l extending generally along a longitudinal axis L, a plate width w extending generally along a transverse axis T, and a primary plate thickness or profile tₚ defined between upper and lowers surfaces 106, 108 of the reinforced spinal plate 100. Additionally, the spinal plate 100 includes an elongate reinforcement portion 110 defining a localized increased plate thickness or profile tᵢ relative to the primary thickness tₚ to strengthen the plate 100, the details of which will be set forth below.

As shown in FIG. 4, the bottom surface 108 of the spinal plate 100 preferably defines a concave curvature C extending axially along the longitudinal axis L, and also preferably defines a similar concave curvature extending laterally along the transverse axis T. The concave curvatures preferably correspond to the anatomical or lordotic curvature of the anterior-facing surfaces of the superior and inferior vertebrae Vₛ, Vᵢ. The upper surface 106 of the plate 100 may define a convex curvature to reduce trauma to the adjacent soft tissue when the reinforced plate 100 is secured to the spinal column. It should be understood that the upper and lower surfaces 106, 108 of the plate 100 may be shaped or contoured to accommodate the specific spinal anatomy and vertebral pathology involved in any particular application of the stabilization system 10.

In one embodiment of the invention, the reinforced spinal plate 100 includes a plurality of openings 120 extending between the upper and lower surfaces 106, 108 and sized to receive the bone screws 102 therethrough for anchoring the plate 100 to the spinal column. In the illustrated embodiment, the reinforced spinal plate 100 includes a pair of laterally offset openings 120a, 120b extending through a first end portion of the plate, and a pair of laterally offset openings 120c, 120d extending through an opposite second end portion of the plate. In one embodiment, the openings 120 are identical in size and configuration, and are symmetrically positioned relative to both the longitudinal axis L and the transverse axis T. However, it should be understood that other sizes, configurations and positions of the openings 120 are also contemplated, and that a single opening or three or more opening may alternatively extend through each end portion of the plate 100 for receiving a corresponding number of the bone screws 102. Each of the openings 120 includes a generally cylindrical bore 122 extending from the lower plate surface 108 and a partially spherical recess 124 extending from the cylindrical bore 122 toward the upper plate surface 106. The partially spherical recess 124 is sized to receive a spherical-shaped head portion (not shown) of the bone anchor 102 therein to allow angulation of the bone anchor 102 relative to the spinal plate 100, the details of which are disclosed in the above-listed U.S. Pat. Nos. 6,293,949 and 6,152,927.

In one embodiment of the present invention, the reinforced spinal plate 100 is formed of a non-metallic material. Such non-metallic materials may comprise polymeric materials including, but not limited to, PEEK (polyetheretherketone), CF-PEEK (carbon fiber/polyetheretherketone), PLA (polylactate) and PLDLA (poly L-lactic/D-L-lactic acid). Other non-metallic materials are also contemplated, including plastic materials, synthetic materials, composite materials, biological materials such as bone tissue, demineralized bone matrix and bone substitute materials, ceramic materials, or any other suitable non-metallic material that would occur to one of skill in the art. Additionally, the reinforced spinal plate 100 may be formed of a resorbable material or a non-resorbable material. Examples of resorbable materials include polylactide, polyglycolide, tyrosine-derived polycarbonate, polyanhydride, polyorthoester, polyphosphazene, calcium phosphate, hydroxyapatite, bioactive glass, and combinations thereof. Examples of non-resorbable materials include non-reinforced polymers, carbon-reinforced polymer composites, PEEK and PEEK composites, ceramics, and combinations thereof. For purposes of the present invention, the term “non-metallic material” includes any material that is not formed entirely of a metallic material, including materials that are formed of a combination of metallic and non-metallic materials.

As indicated above, spinal plates formed of a non-metallic materials generally have reduced mechanical properties (e.g., strength, rigidity, etc.) compared to spinal plates formed of metallic materials. In order to compensate for reduced mechanical properties, the overall thickness of the spinal plate may be uniformly increased, thereby resulting in the use of a greater volume of plate material. However, the use of a greater amount of plate material may not be desirable in instances where the spinal plate is formed of a resorbable material as this would significantly add to the volume of material to be resorbed into the body, and more particularly into the paraspinal soft tissues. Additionally, the use of a greater volume of plate material may also increase the overall size, weight and profile of the spinal plate, the likes of which may be problematic in instances where such properties and characteristics should preferably be minimized.

The reinforced spinal plate 100 of the present invention avoids the disadvantages associated with uniformly increasing the overall plate thickness by minimizing the primary thickness tₚ of the plate material or profile while providing the spinal plate 100 with one or more elongate reinforcement portions 110 that define a localized increased thickness tᵢ in plate material or profile to provide additional strength or reinforcement to the spinal plate 100. As a result, the structural integrity and/or rigidity of the reinforced spinal plate 100 is enhanced. As should be appreciated, the spinal plate 100 is strengthened by varying plate geometry and/or adding geometric features that reinforce the plate as opposed to increasing the overall plate thickness or profile. The enhanced structural integrity and/or rigidity of the reinforced spinal plate 100 in turn results in an increased capability of resisting greater levels of bending stresses, torsional loading, compression loading and/or tension loading exerted onto the reinforced spinal plate 100 by the vertebrae Vₛ, Vᵢ. In one embodiment of the invention, the localized increased thickness tᵢ in plate material or profile is at least about twenty-five percent larger than the primary thickness tₚ in plate material or profile. In another embodiment of the invention, the localized increased thickness tᵢ in plate material or profile is at least about fifty percent larger than the primary thickness tₚ in plate material or profile.

As shown most clearly in FIGS. 2 and 4, the elongate reinforcement portion 110 of the spinal plate 100 which defines the localized increased thickness tᵢ in plate material or profile extends in a lateral direction generally along the transverse axis T and the plate width w, and is centrally positioned between the laterally offset openings 120a, 120c and 120b, 120d. In the illustrated embodiment, the elongate reinforcement portion 110 comprises an elongate surface projection or ridge projecting outwardly from the upper or anteriorly-facing surface 106 of the spinal plate 100 and extending along the entire width w of the reinforced spinal plate 100. However, it should be understood that in other embodiments of the invention, the elongate reinforcement portion 110 may project from the lower or posteriorly-facing surface 108, may extend along less than the entire plate width w, and may be positioned along other regions of the reinforced spinal plate 100.

In the illustrated embodiment, the elongate reinforcement portion 110 is formed integral with the remainder of the spinal plate 100 to define a unitary, single-piece plate structure. However, it should be understood that in other embodiments of the invention, the elongate reinforcement portion 110 may be formed separately from the remainder of the plate and subsequently attached to the upper plate surface 106 via an attachment technique including, but not limited to, welding, bonding, fastening or any other suitable attachment technique know to those of skill in the art. The outer surface 112 of the elongate reinforcement portion 110 is preferably convexly curved or rounded and sharp edges or corners are minimized so that the surrounding soft tissues do not encounter high profile, aggressive or sharp protrusions that may lead to tissue trauma or dysphagia.

As should be appreciated, a significant portion of the reinforced spinal plate 100 has a substantially uniform primary plate thickness tₚ defined between the upper and lowers surfaces 106, 108. In the illustrated embodiment, the localized increased thickness tᵢ in plate material or profile is confined to the central or mid-portion of the reinforced spinal plate 100 and extends in a lateral or horizontal direction (e.g., along the coronal plane) when the reinforced spinal plate 100 is anchored to the upper and lower vertebrae Vₛ, Vᵢ. As should also be appreciated, by limiting the localized increased thickness tᵢ in plate material or profile to a select portion or region of the reinforced spinal plate 100, while maintaining the primary plate thickness tₚ for the remainder of the spinal plate 100, the volume of plate material is minimized. As should further be appreciated, the localized increased thickness tᵢ in plate material or profile defined by the elongate reinforcement portion 110 increases plate strength and rigidity along the central region of the plate where relatively high bending moments or loads are typically experienced. The elongate reinforcement portion 110 also increases plate strength and rigidity to resist torsional loading, compression loading and/or tension loading exerted onto the reinforced spinal plate 100 by the vertebrae Vₛ, Vᵢ. The geometric design of the reinforced spinal plate 100 therefore enhances structural integrity and/or rigidity while minimizing the volume of material used to form the plate.

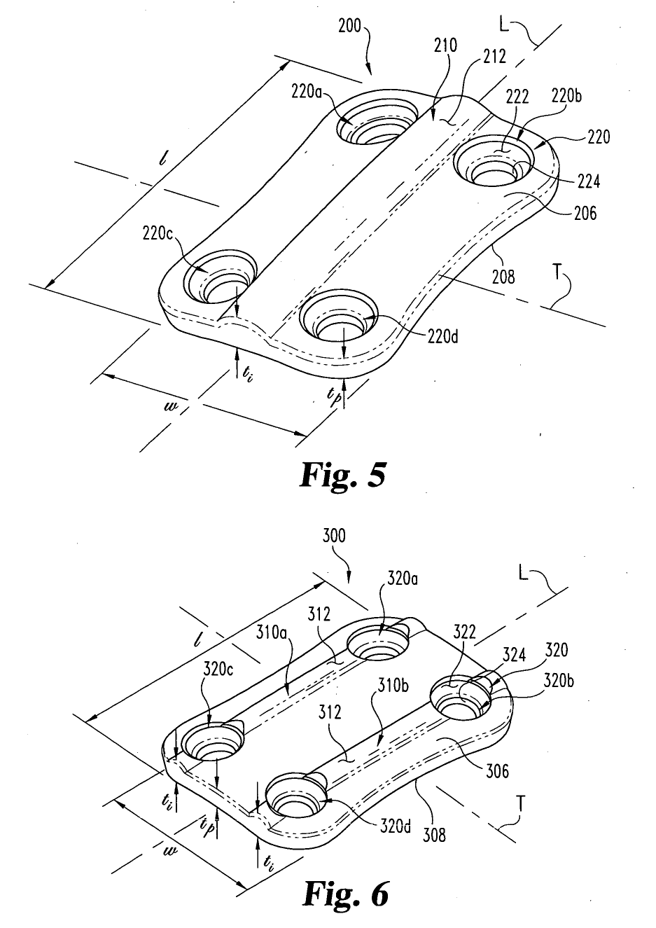

Referring now to FIG. 5, shown therein is another embodiment of a reinforced spinal plate 200 for use in association with a spinal stabilization system. Like the reinforced spinal plate 100, the reinforced spinal plate 200 is formed of a non-metallic material, and may be formed of a resorbable material or a non-resorbable material. Additionally, the reinforced spinal plate 200 is sized to span a distance between superior and inferior vertebrae Vₛ, Vᵢ, and is likewise configured to be secured to the vertebrae via a plurality of bone anchors, such as, for example, bone screws 102. The reinforced spinal plate 200 has a plate length l extending generally along a longitudinal axis L, a plate width w extending generally along a transverse axis T, and a primary plate thickness or profile tₚ defined between upper and lowers surfaces 206, 208 of the plate 200. Additionally, the reinforced spinal plate 200 includes an elongate reinforcement portion 210 defining a localized increased plate thickness or profile tᵢ relative to the primary thickness tₚ to provide additional strength to the plate 200, the details of which will be set forth below.

The bottom surface 208 of the reinforced spinal plate 200 preferably defines a concave curvature extending axially along the longitudinal axis L and also preferably defines a similar concave curvature extending laterally along the transverse axis T, with each of the concave curvatures preferably corresponding to the anatomical or lordotic curvature of the anterior-facing surfaces of the superior and inferior vertebrae Vₛ, Vᵢ. The upper surface 206 of the plate 200 may define a convex curvature to reduce the amount of trauma to the adjacent soft tissue when the spinal plate 200 is secured to the spinal column. In one embodiment of the invention, the reinforced spinal plate 200 includes a plurality of openings 220 extending between the upper and lower surfaces 206, 208 and sized to receive the bone screws 102 therethrough for anchoring the plate 200 to the spinal column.

In the illustrated embodiment, the reinforced spinal plate 200 includes a pair of laterally offset openings 220a, 220b extending through a first end portion of the plate, and a pair of laterally offset openings 220c, 220d extending through an opposite second end portion of the plate. In one embodiment, the openings 220 are identical in size and configuration, and are symmetrically positioned relative to both the longitudinal axis L and the transverse axis T. However, it should be understood that other sizes, configurations and positions of the openings 220 are also contemplated, and that a single opening or three or more opening may alternatively extend through each end portion of the plate 200 for receiving a corresponding number of the bone screws 102. Each of the openings 220 includes a generally cylindrical bore 222 extending from the lower plate surface 208 and a partially spherical recess 224 extending from the cylindrical bore 222 toward the upper plate surface 206. The partially spherical recess 224 is sized to receive a spherical-shaped head portion of the bone screw 102 therein to allow angulation of the bone anchor 102 relative to the reinforced spinal plate 200.

The elongate reinforcement portion 210 defines a localized increased thickness tᵢ in plate material or profile to strengthen the spinal plate 200, which in turn results in an increased capability of resisting greater levels of bending stresses, torsional loading, compression loading and/or tension loading exerted onto the reinforced spinal plate 200 by the vertebrae Vₛ, Vᵢ. In the illustrated embodiment of the reinforced spinal plate 200, the elongate reinforcement portion 210 extends in a direction generally along the longitudinal axis L and the plate length l (e.g., in a superior-inferior direction), and is centrally positioned between the pairs of laterally offset openings 220a, 220b and 220c, 220d. The elongate reinforcement portion 210 comprises an elongate surface projection or ridge projecting outwardly from the upper or anteriorly-facing surface 206 of the spinal plate 200 and extending along the entire length l of the reinforced spinal plate 200. However, it should be understood that in other embodiments of the invention, the elongate reinforcement portion 210 may project from the lower or posteriorly-facing surface 208, may extend along less than the entire plate length l, and may be positioned along other regions of the reinforced spinal plate 200.

In the illustrated embodiment, the elongate reinforcement portion 210 is formed integral with the remainder of the reinforced spinal plate 200 to define a unitary, single-piece plate structure. However, it should be understood that in other embodiments of the invention, the elongate reinforcement portion 210 may be formed separately from the remainder of the plate and subsequently attached to the upper plate surface 206 via an attachment technique including, but not limited to, welding, bonding, fastening or any other suitable attachment technique know to those of skill in the art. The outer surface 212 of the elongate reinforcement portion 210 is preferably convexly curved or rounded and sharp edges or corners are minimized so that the surrounding soft tissues do not encounter high profile, aggressive or sharp protrusions that may lead to tissue trauma or dysphagia.

As should be appreciated, a significant portion of the reinforced spinal plate 200 has a substantially uniform primary plate thickness tₚ defined between upper and lowers surfaces 206, 208. In the illustrated embodiment, the localized increased thickness tᵢ in plate material or profile is confined to a central or mid-portion of the reinforced spinal plate 200 and extends in an axial or vertical direction (e.g., along the sagittal plane) when the reinforced spinal plate 200 is anchored to the upper and lower vertebrae Vₛ, Vᵢ. As should also be appreciated, by limiting the localized increased thickness tᵢ in plate material or profile to a select portion or region of the reinforced spinal plate 200, while maintaining the primary plate thickness tₚ for the remainder of the spinal plate 200, the volume of plate material is minimized. As should further be appreciated, the localized increased thickness tᵢ in plate material or profile extending axially along a central or mid-portion of the spinal plate 200 increases plate strength and rigidity adjacent the central region of the plate where relatively high bending moments or loads are typically experienced. The elongate reinforcement portion 210 also increases plate strength and rigidity to resist torsional loading, compression loading and/or tension loading exerted onto the reinforced spinal plate 200 by the vertebrae Vₛ, Vᵢ.

Referring now to FIG. 6, shown therein is another embodiment of a reinforced spinal plate 300 for use in association with a spinal stabilization system. Like the reinforced spinal plates 100 and 200, the reinforced spinal plate 300 is formed of a non-metallic material, and may be formed of a resorbable material or a non-resorbable material. Additionally, the reinforced spinal plate 300 is sized to span a distance between superior and inferior vertebrae Vₛ, Vᵢ, and is likewise configured to be secured to the vertebrae via a plurality of bone anchors, such as, for example, the bone screws 102. The reinforced spinal plate 300 has a plate length l extending generally along a longitudinal axis L, a plate width w extending generally along a transverse axis T, and a primary plate thickness or profile tₚ defined between upper and lowers surfaces 306, 308 of the plate 300. Additionally, the reinforced spinal plate 300 includes a pair of elongate reinforcement portions 310a, 310b, each defining localized increased plate thickness or profile tᵢ relative to the primary thickness tₚ to provide additional strength to the plate 300, the details of which will be set forth below.

The bottom surface 308 of the reinforced spinal plate 300 preferably defines a concave curvature extending axially along the longitudinal axis L and also preferably defines a similar concave curvature extending laterally along the transverse axis T, with each of the concave curvatures preferably corresponding to the anatomical or lordotic curvature of the anterior-facing surfaces of the superior and inferior vertebrae Vₛ, Vᵢ. The upper surface 306 of the plate 300 may define a convex curvature to reduce the amount of trauma to the adjacent soft tissue when the spinal plate 300 is secured to the spinal column. In one embodiment of the invention, the reinforced spinal plate 300 includes a plurality of openings 320 extending between the upper and lower surfaces 306, 308 and sized to receive the bone screws 102 therethrough for anchoring the plate 300 to the spinal column.

In the illustrated embodiment, the reinforced spinal plate 300 includes a pair of laterally offset openings 320a, 320b extending through a first end portion of the plate, and a pair of laterally offset openings 320c, 320d extending through an opposite second end portion of the plate. In one embodiment, the openings 320 are identical in size and configuration, and are symmetrically positioned relative to both the longitudinal axis L and the transverse axis T. However, it should be understood that other sizes, configurations and positions of the openings 320 are also contemplated, and that a single opening or three or more opening may alternatively extend through each end portion of the plate 300 for receiving a corresponding number of the bone screws 102. Each of the openings 320 includes a generally cylindrical bore 322 extending from the lower plate surface 308 and a partially spherical recess 324 extending from the cylindrical bore 322 toward the upper plate surface 306. The partially spherical recess 324 is sized to receive a spherical-shaped head portion of the bone screw 102 therein to allow angulation of the bone anchor 102 relative to the reinforced spinal plate 300.

The elongate reinforcement portions 310a, 310b each define a localized increased thickness tᵢ in plate material or profile to strengthen the spinal plate 300, which in turn results in an increased capability of resisting greater levels of bending stresses, torsional loading, compression loading and/or tension loading exerted onto the spinal plate 300 by the vertebrae Vₛ, Vᵢ. In the illustrated embodiment of the reinforced spinal plate 300, the elongate reinforcement portions 310a, 310b each extend in a direction generally along the longitudinal axis L and the plate length l (e.g., in a superior-inferior direction) with the elongate reinforcement portion 310a extending between the pairs of laterally offset openings 320a, 320c and the elongate reinforcement portion 310b extending between the pairs of laterally offset openings 320b, 320d. The elongate reinforcement portions 310a, 310b each comprise an elongate surface projection or ridge projecting outwardly from the upper or anteriorly-facing surface 306 of the spinal plate 300 and extending along the entire length l of the reinforced spinal plate 300. However, it should be understood that in other embodiments of the invention, the elongate reinforcement portions 310a, 310b may project from the lower or posteriorly-facing surface 308, may extend along less than the entire plate length l, and may be positioned along other regions of the reinforced spinal plate 300.

In the illustrated embodiment, the elongate reinforcement portions 310a, 310b are formed integral with the remainder of the spinal plate 300 to define a unitary, single-piece plate structure. However, it should be understood that in other embodiments of the invention, the elongate reinforcement portions 310a, 310b may be formed separately from the remainder of the plate and subsequently attached to the upper plate surface 306 via an attachment technique including, but not limited to, welding, bonding, fastening or any other suitable attachment technique know to those of skill in the art. The outer surfaces 312 of the elongate reinforcement portions 310a, 310b 310 are preferably convexly curved or rounded and sharp edges or corners are minimized so that the surrounding soft tissues do not encounter high profile, aggressive or sharp protrusions that may lead to tissue trauma or dysphagia.

As should be appreciated, a significant portion of the reinforced spinal plate 300 has a substantially uniform primary plate thickness tₚ defined between upper and lowers surfaces 306, 308. In the illustrated embodiment, the localized increased thickness tᵢ in plate material or profile is confined to a pair of laterally offset regions of the reinforced spinal plate 300 which extend in an axial or vertical direction (e.g., along the sagittal plane) when the reinforced spinal plate 300 is anchored to the upper and lower vertebrae Vₛ, Vᵢ. As should also be appreciated, by limiting the localized increased thickness tᵢ in plate material or profile to select portions or regions of the reinforced spinal plate 300, while maintaining the primary plate thickness tₚ for the remainder of the spinal plate 300, the volume of plate material is minimized. As should also be appreciated, the localized increased thickness tᵢ in plate material or profile extending along axial side portions of the reinforced spinal plate 300 increases plate strength and rigidity of the plate. The elongate reinforcement portions 310a, 310b also increase plate strength and rigidity to resist torsional loading, compression loading and/or tension loading exerted onto the reinforced spinal plate 300 by the vertebrae Vₛ, Vᵢ.

In still other embodiments of the invention, reinforced spinal plates may be provided which are formed of a non-metallic material and which include one or more elongate reinforcement portions that define a localized increased plate thickness or profile tᵢ relative to the primary thickness tₚ of the plate. The elongate reinforcement portions may extend axially along the length l of the plate, laterally across the width w of the plate, or diagonally along/across the plate at any angle relative to the longitudinal or transverse axes of the plate. Additionally, the elongate reinforcement portions may have a linear configuration, an angled configuration, a curved/curvilinear configuration, a circular or oval-shaped configuration, or any combination thereof. Further, the reinforced spinal plate may include two or elongate reinforcement portions that extend in the same directions or in different directions. In one embodiment, one of the elongate reinforcement portions may extend along the plate length with the other extending across the plate width so as to define a cross-shaped configuration. In another embodiment, two elongate reinforcement portions may extend diagonally between opposite corners of the spinal plate so as to define an X-shaped configuration. In still another embodiment, elongate reinforcement portions may be provided which define a diamond-shaped configuration. Other suitable configurations of the elongate reinforcement portion(s) are also contemplated as would occur to one of skill in the art.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. An orthopedic stabilization device, comprising:
a plate formed of a non-metallic material and defining a primary thickness profile, said plate including an elongate reinforcement portion defining a localized increased thickness profile to strengthen said plate.

2. The device of claim 1, wherein said non-metallic material comprises a polymeric material.

3. The device of claim 2, wherein said polymeric material is selected from the group consisting of PEEK (polyetheretherketone), CF-PEEK (carbon fiber/polyetheretherketone), PLA (polylactate) and PLDLA (poly L-lactic/D-L-lactic acid).

4. The device of claim 1, wherein said non-metallic material comprises a resorbable material.

5. The device of claim 1, wherein said elongate reinforcement portion comprises an elongate surface projection extending from at least one of an upper and lower surface of said plate.

6. The device of claim 5, wherein said elongate surface projection comprises an elongate ridge.

7. The device of claim 1, wherein said elongate reinforcement portion is formed integral with said plate to define a unitary, single-piece plate structure.

8. The device of claim 1, wherein said elongate reinforcement portion extends along one of a length dimension and a width dimension of said plate.

9. The device of claim 8, wherein said elongate reinforcement portion extends entirely along said one of said length dimension and said width dimension of said plate

10. The device of claim 1, wherein said plate includes a plurality of openings extending through said thickness profile for receiving bone anchors to anchor said plate to bone.

11. The device of claim 10, wherein said plate extends along a longitudinal axis and includes a first pair of said openings laterally offset relative to one another and extending through said thickness profile adjacent a first end portion of said plate, said plate including a second pair of said openings laterally offset relative to one another and extending through said thickness profile adjacent an opposite second end portion of said plate, said elongate reinforcement portion extending laterally across a width of said plate and positioned between said first and second pairs of said openings.

12. The device of claim 10, wherein said plate extends along a longitudinal axis and includes a first pair of said openings laterally offset relative to one another and extending through said thickness profile adjacent a first end portion of said plate, said plate including a second pair of said openings laterally offset relative to one another and extending through said thickness profile adjacent an opposite second end portion of said plate, said elongate reinforcement portion extending axially along a length of said plate and positioned between said laterally offset openings of said first and second pairs of said openings.

13. The device of claim 10, wherein said plate extends along a longitudinal axis and includes a first pair of said openings laterally offset relative to one another and extending through said thickness profile adjacent a first end portion of said plate, said plate including a second pair of said openings laterally offset relative to one another and extending through said thickness profile adjacent an opposite second end portion of said plate, said plate including first and second elongate reinforcement portions laterally offset relative to one another and extending axially along a length of said plate, said first elongate reinforcement portion extending between a first opening of said first pair of openings and a second opening of said second pair of openings, said second elongate reinforcement portion extending between a third opening of said first pair of openings and a forth opening of said second pair of openings.

14. The device of claim 13, wherein said first and second elongate reinforcement portions are arranged generally parallel to one another.

15. The device of claim 1, wherein said elongate reinforcement portion extends along a central region of said plate.

16. The device of claim 1, wherein said elongate reinforcement portion includes an outwardly facing surface defining a convex curvature.

17. The device of claim 1, wherein said localized increased thickness profile is at least about twenty-five percent larger than said primary thickness profile.

18. The device of claim 17, wherein said localized increased thickness profile is at least about fifty percent larger than said primary thickness profile.

19. The device of claim 1, wherein said plate is formed entirely of said non-metallic material.

20. The device of claim 1, wherein said plate includes a pair of said elongate reinforcement portions, each of said elongate reinforcement portions defining a localized increased thickness profile relative to said primary thickness profile.

21. The device of claim 20, wherein said pair of elongate reinforcement portions are offset from one another and arranged generally parallel to one another.

22. The device of claim 20, wherein said pair of elongate reinforcement portions extend along a length dimension of said plate.

23. The device of claim 20, wherein one of said elongate reinforcement portions extends along a length dimension of said plate and another of said elongate reinforcement portions extends along a width dimension of said plate.

24. The device of claim 20, wherein said elongate reinforcement portions extend diagonally between opposite corners of said plate to define an X-shaped configuration.

25. An orthopedic stabilization device, comprising:
a plate formed of a non-metallic material and defining a primary material thickness, said plate including an elongate reinforcement portion defining a localized increased material thickness to strengthen said plate.

26. The device of claim 25, wherein said non-metallic material comprises a polymeric material.

27. The device of claim 25, wherein said non-metallic material comprises a resorbable material.

28. The device of claim 25, wherein said elongate reinforcement portion comprises an elongate surface projection extending from at least one of an upper and lower surface of said plate.

29. The device of claim 25, wherein said elongate surface projection comprises an elongate ridge.

30. The device of claim 25, wherein said elongate surface projection is formed integral with said plate to define a unitary, single-piece plate structure.

31. The device of claim 25, wherein said elongate reinforcement portion extends along one of a length dimension and a width dimension of said plate.

32. The device of claim 25, wherein said plate is formed entirely of said non-metallic material.

33. The device of claim 25, wherein said localized increased thickness profile is at least about twenty-five percent larger than said primary thickness profile.

34. The device of claim 33, wherein said localized increased material thickness is at least about fifty percent larger than said primary material thickness.

35. An orthopedic stabilization device, comprising:
a plate formed of a non-metallic material and defining a primary material thickness between oppositely-facing first and second surfaces, said plate including at least one elongate surface projection formed integral with said plate to define a unitary, single-piece plate structure and extending along a dimension of one of said first and second surfaces to strengthen said plate.

36. The device of claim 35, wherein said non-metallic material comprises a polymeric material.

37. The device of claim 35, wherein said non-metallic material comprises a resorbable material.

38. The device of claim 35, wherein said elongate surface projection comprises an elongate ridge.

39. The device of claim 35, wherein said plate is formed entirely of said non-metallic material

40. The device of claim 35, wherein said elongate reinforcement portion extends entirely along said dimension of said one of said first and second surfaces.

41. The device of claim 35, wherein said dimension comprises a length dimension.

42. The device of claim 35, wherein said dimension comprises a width dimension.

43. The device of claim 35, wherein said localized increased thickness profile is at least about twenty-five percent larger than said primary thickness profile.

44. The device of claim 43, wherein said localized increased material thickness is at least about fifty percent larger than said primary material thickness.

45. The device of claim 35, wherein said elongate surface projection includes an outwardly facing surface defining a convex curvature.
